(19) United States
(12) Patent Application Publication
(10) Pub. No.: US 2015/0032190 A1
(43) Pub. Date: Jan. 29, 2015

(54) METHODS AND APPARATUS FOR OMNIDIRECTIONAL TISSUE ILLUMINATION

(71) Applicant: Leah Christine Acker, Cambridge, MA (US)

(72) Inventor: Leah Christine Acker, Cambridge, MA (US)

(73) Assignee: Massachusetts Institute of Technology, Cambridge, MA (US)

(21) Appl. No.: 14/337,938

(22) Filed: Jul. 22, 2014

Related U.S. Application Data
(60) Provisional application No. 61/857,440, filed on Jul. 23, 2013.

(51) Int. Cl. A61N 5/06; F21V 8/00; B29D 11/00

(52) U.S. Cl. CPC A61N 5/0613; B29D 11/00663; B29D 11/00932; G02B 6/0008; A61N 2005/063
USPC 607/88; 264/1.24; 216/24; 362/558

(57) ABSTRACT

Embodiments of the present invention include a fiber-optic tissue illuminator suitable for illuminating large areas of central and peripheral neural tissue, e.g., in a primate brain. Certain examples of the tissue illuminator have a light delivery surface that may be about two orders of magnitude larger than that of a conventional optical fiber of equal diameter. This illuminator allows for substantially more light to be delivered to brain tissue with no more penetration damage than a conventional fiber. For example, an illustrative illuminator can deliver light over a length of at least 3 mm in neural tissue, such as a macaque cortex, as shown by the presence of a light artifact in the local field potential. An exemplary illuminator can also be used with a previously injected viral vector (e.g., halorhodopsin) of optogenetic applications, like silencing neurons distributed over an extended region (e.g., a 3 mm length) of neural tissue.

### CROSS REFERENCE TO RELATED APPLICATION

This application claims priority under 35 U.S.C. 119 to U.S. Application No. 61/857,440, filed on Jul. 23, 2013, and entitled “Methods and Apparatus for Omnidirectional Tissue Illumination,” which is incorporated herein by reference in its entirety.

### GOVERNMENT SUPPORT

This invention was made with government support under Grant No. EY017921 awarded by the National Institutes of Health. The government has certain rights in the invention.

### BACKGROUND

Optogenetics can be used to change the potential of cells, including neurons, cardiac cells, muscle cells, etc. Optogenetics encompasses neuromodulation techniques that use light-sensitive proteins and targeted illumination for real-time control and (indirect) measurement of the activity of individual neurons in living tissue. Optogenetic techniques provide fine spatial, genetic, and temporal resolution, enabling modulation of individual neurons on a millisecond time scale. Optogenetic manipulation can also be used to alter the activity of specific neural areas without directly affecting a subject's behavior.

To modulate neuronal behavior using optogenetic techniques, the targeted neurons are genetically modified by transfection, viral transduction, or transgenically to express light-sensitive proteins that drive or inhibit spiking Once the targeted neurons have begun expressing the desired light-sensitive proteins, the proteins are activated by targeted illumination. In other cell types, these proteins more generally lead to influx or efflux of ions as they are ion channels or ion pumps. For in vivo applications, an optical probe may be used to illuminate the targeted cells with light that activates the proteins. Changing the intensity and wavelength of the illumination modulates the protein, which switches the targeted cells on and off. In theory, the change in potential could drive some other cellular process (e.g., release of vesicles containing a biochemical or up-regulation of some gene, etc.).

For example, optogenetic modulation can be used to change the membrane voltage potential of a neuron, which causes the neuron to “spike,” or transmit a transient electrical signal to one or more neighboring cells. Spiking can be induced by illuminating a light-sensitive, chemically modified ligand that binds exogeneous receptors. Spiking can also be induced by illuminating a transmembrane protein, such as an opsin, that becomes active when illuminated with light at a particular wavelength. Optogenetic modulation can also be used to inhibit signaling between or among neurons, e.g., via membrane hyperpolarization using light-activated proteins like halorhodopsin.

### SUMMARY

Embodiments of the present disclosure include an apparatus for providing diffuse illumination. In one example, the apparatus comprises a multimode optical fiber with a proximal end and a distal end, which terminates in a tapered tip that defines a roughened outer surface. In operation, the proximal end couples light into the multimode optical fiber. The distal end emits at least a portion of the light coupled into the multimode optical fiber via the roughened outer surface as diffuse illumination.

In at least one example, the multimode optical fiber comprises a polymer cladding disposed about a polymer core to guide the light from the proximal end to the distal end. The multimode optical fiber may have a stiffness of about 1 GPa to about 100 GPa.

The tapered tip may have a maximum outer diameter of about 40 microns to about 5 millimeters and a length of about 1 millimeter to about 10 millimeters. In some cases, the tapered tip defines an apex forming an angle of about 1 degree to about 30 degrees (e.g., about 10 degrees). The tapered tip may be configured to be inserted into tissue (e.g., neural tissue or other tissue or into cavities within the body) at a reduced risk of damaging the tissue (e.g., without damaging vasculature in the tissue). The tapered tip may also be configured to emit the diffuse illumination over a solid angle of about π/8 steradians to about π/2 steradians.

The roughened outer surface may be formed by sanding at least a portion of the tapered tip with sandpaper having a grit of about 1 micron to about 5 microns. For example, the roughened outer surface may extend over an area of about 0.1 mm² to about 100 mm² (e.g., from about 1 mm² to about 10 mm²).

Some examples of the apparatus may also include a light source that is optically coupled to the proximal end of the multimode fiber. In operation, the light source emits the light into the multimode optical fiber. In some cases, the light source is configured to emit the light at a wavelength selected to modulate at least one light-activated particle (e.g., a protein or other molecule) disposed within an animal body or within a crevis on the surface of an animal body (e.g., between the toes in the case of diabetic ulcers or fungal infections, between the teeth in dentistry, between the folds of skin patients (especially morbidly obese patients) afflicted with cellulitis). For instance, the light source may modulate fluorescence emission from a light-activated molecule or resonant absorption and heating by a nanoparticle. The light source may also be configured to emit the light at a wavelength absorbed by tissue in an animal body or at a wavelength used to cure an optically curable adhesive, such an ultraviolet (UV) curable adhesive, or to heat or irradiate unaltered tissue (e.g., with UV light).

Other embodiments include a method of using an optical fiber to emit diffuse illumination within an animal body. Such a method may include inserting the distal end of an optical fiber into the animal body. Light is coupled into a proximal end of the optical fiber and guided from the proximal end to the distal end via the optical fiber. At the distal end, which terminates in a tapered tip having a roughened outer surface, at least a portion of the light is emitted, via the roughened surface of the tapered tip, within the animal body as diffuse illumination.

In some instances, the distal end is inserted into neural tissue of the animal body without significantly or substantially damaging vasculature in and/or surrounding the neural tissue. For example, the tapered tip may be guided to a desired location within the neural tissue. When positioned as desired, the wavelength and/or intensity of the light coupled into the proximal end of the optical fiber may be modulated so as to modulate at least one light-activated protein illuminated by the diffuse illumination. Any resulting change in expression of at least one cell (e.g., a neuron) in response to modulation of the at least one light-activated protein can be recorded. For instance, the diffuse illumination may be emitted over a solid angle of about π/8 steradians to about π/2 steradians, through an area of the roughened surface of about 1 mm² to about 10 mm², and/or over an area within the animal body having a length of about 1 millimeter to about 10 millimeters.

In another instance, the distal end may be inserted into a lumen in the animal body and used to illuminate at least a portion of an interior surface of the lumen. In such an instance, the light coupled into the fiber's proximal may be generated at a wavelength absorbed by at least one of tissue of the lumen, plaque deposited on an interior surface of the lumen, a thrombus, a foreign object disposed within the animal body, a liquid injected into the animal body implanted material, a weakened region of tissue, scar tissue, a region of tissue deformation, and/or a cavity within the animal body. The diffuse illumination may also be used to facilitate acquisition of at least one image of some or all of the lumen's interior surface.

Yet another embodiment comprises a method of making a fiber-optic illuminator from an optical fiber. First, a distal end of the optical fiber is formed into a tapered tip, e.g., by softening the distal end of the optical fiber and drawing the distal end of the optical fiber so as to form the tapered tip. In some cases, the tapered tip may have a maximum outer diameter of about 40 microns to about 5 millimeters and a length of about 1 millimeter to about 10 millimeters. At least a portion of the tapered tip is roughened so as to form a roughened outer surface that emits diffuse illumination when light is coupled into a proximal end of the optical fiber. For instance, the tip may be roughened by chemical etching, laser etching, thermal etching, and/or mechanical abrasion, e.g., by sanding the at least a portion of the tapered tip with sandpaper (e.g., diamond grit paper or ruby grit paper) having a grit of about 1 micron to about 5 microns.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is a color photograph of expression of a viral vector containing a halorhodopsin (AAV8-hSyn-JAWS-GFP) across all layers of macaque somatosensory cortex.

FIG. 2A is a diagram of an optical setup that includes an omnidirectional tissue illuminator optically coupled to a laser via a shutter.

FIG. 2B is a diagram of an optical fiber coupled to an omnidirectional tissue illuminator with a mating sleeve suitable for use in the optical setup of FIG. 2B.

FIG. 2C shows a longitudinal cross section of an omnidirectional tissue illuminator.

FIG. 2D shows a radial cross section of an optical fiber.

FIG. 2E shows a radial cross section of the tip of an omnidirectional tissue illuminator.

FIGS. 2F-2L show tissue illuminators with a variety of shaped tips.

FIG. 3A is a photograph of the etched tip of a penetrating omnidirectional tissue illuminator with laser light emitted via a 5 mm length without cladding.

FIG. 3B is a photograph of the etched tip shown in FIG. 3A that shows a taper from a diameter of about 250 μm to about 60-100 μm.

FIG. 3C is a photograph of a penetrating omnidirectional tissue illuminator inserted into a block of agar.

FIGS. 4A-4C are photographs of tissue illuminators with conical, angled, and etched tips.

FIG. 5A is a flow chart that illustrates a process of making a penetrating omnidirectional tissue illuminator.

FIG. 5B is a flow chart that illustrates a fabrication process for an etched primate tissue illuminator.

FIG. 6A illustrates a measurement of the total optical power emitted from the tip of an omnidirectional tissue illuminator with an integrating sphere.

FIG. 6B is a cross-sectional view of the integrating sphere and omnidirectional tissue illuminator shown in FIG. 6A.

FIG. 6C is a plot of optical power emitted by a representative omnidirectional tissue illuminator versus insertion depth using the integrating sphere shown in FIGS. 4A and 4B.

FIG. 7A is a false-color magnetic resonance image of illumination and recording sites in the cortex that shows the grid through which the electrodes and the illuminator are lowered and the target region (white box).

FIG. 7B is an enlarged view of the target region shown in FIG. 7A with the trajectories of the illuminator and electrode shown as well as the virus injection region and the locations of the sample neurons shown in FIG. 8.

FIG. 8 includes plots of the local field potential (LFP) recordings of representative neurons (left column) at the locations shown in FIGS. 7A and 7B and instantaneous spike time histograms (right column).

### DETAILED DESCRIPTION

Embodiments of the present invention include a flexible, biocompatible omnidirectional tissue illuminator suitable for optogenetic neuronal modulation in primates and other animals. In one example, the tissue illuminator includes an optical fiber that terminates in a tapered distal tip with a roughened outer surface. The optical fiber guides light from a light source, such as a laser, to the distal tip, which emits the light via the roughened outer surface as uniform, diffuse illumination. The roughened surface may extend over a length of about 1 mm to about 10 mm (e.g., about 5 mm), which enables the illuminator to deliver (or receive) light over an area that is several times larger than the fiber's cross-sectional area. (Conversely, conventional optical fibers emit light from a flat or slightly modified tip with an emitting surface whose area is roughly equal to the fiber's cross-sectional area.)

The tissue illuminator's distal tip may be drawn or tapered from one end of a polymer optical fiber, then etched or abraded to define an outer surface that emits light guided by the fiber as diffuse illumination. In some cases, the fiber's core and cladding may be fused together during the drawing process to form a tip with a homogeneous or relatively smooth refractive index profile instead of a step index profile like the fiber. In other cases, the cladding may be thinned so much or removed such that the distal tip may have a smooth refractive index profile. Because the tip lacks a step index profile, it does not guide light; instead, it allows light to diverge over a range of angles spanning the fiber's numerical aperture (given by the square root of the difference between the squares of the core and cladding indices). Roughening the tip, e.g., by sanding it with 3-5 μm sandpaper, creates an irregular surface that diffuses the diverging beam. Because the tip's outer surface is roughened, the emitted beam does not include any hot spots (regions of high intensity) that might otherwise heat or damage the surrounding tissue. The tip's large surface area also allows light to reach more cells directly instead of propagating through surrounding tissue, which further reduces tissue heating.

In addition, the illuminator's tapered shape, thin tip, and flexibility reduce penetration damage, which is particularly beneficial for biological and medical applications. Some embodiments of the tissue illuminator may allow for a smaller radius of penetration, and thus less damage (e.g., smaller penetration diameter to light emitting surface ratio) than any conventional fiber-based illuminator. This allows for more acute penetrations in a particular brain area before tissue becomes too degraded to continue recording. Illuminators made of biocompatible plastics and polymers, such as poly (methyl methacrylate) (PMMA), may be less likely to break inside the tissue than conventional optical fibers, which are made of silica or glass. As a result, plastic- and polymer-based illuminators tend to be safer to use in biological tissue, such as central or peripheral neural tissue, than glass or silica illuminators.

An acrylic illuminator can also be placed chronically in the brain with fewer complications than expected with a glass or silica optical fiber. Evidence suggests that a more flexible electrode (e.g., Tungsten microwires) causes less tissue damage than a rigid electrode do because it exerts less shear stress and tearing as the it moves relative to the brain. Similarly, a flexible plastic illuminator may cause less damage chronically than a rigid glass fiber, especially when implanted chronically. It can also be used as a light source for imaging the location of a probe coupled to the fiber through the skin, for providing light during endoscopic or laparoscopic surgical procedures, or for activating fluorescent proteins.

Omnidirectional Tissue Illumination for Optogenetics

Applications for inventive omnidirectional tissue illuminators include, but are not limited to modulating behavior using optogenetics, e.g., in primates. More specifically, a possible application is in neuroscience, where an exemplary illuminator could be used to deliver light across all layers of the primate cortex with a single penetration. This illumination, in conjunction with optogenetics techniques, would allow for neuromodulation of large brain regions, including regions of the cortex, pulvinar, amygdala, and subthalamic nuclei.

While optogenetics is widely used in the rodent neuroscience community, to date it has been used only a handful of times in primates and, then, primarily in proof-of-concept studies. Much of the challenge of adapting optogenetics to the primate may be a function of brain size. For instance, the macaque brain is more than 200 times larger than the mouse brain. Similarly, the tissue volume of a region of interest in the macaque brain may be two orders of magnitude larger than an analogous volume in the mouse.

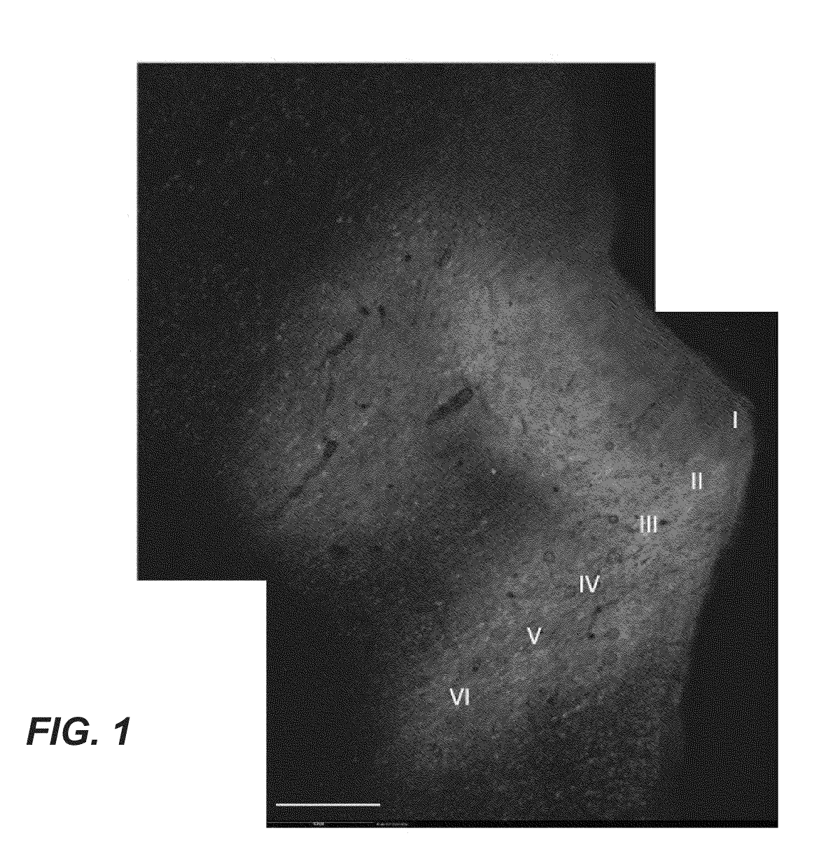

FIG. 1 illustrates the size of a region of interest in a macaque somatosensory cortex. It shows expression of a viral vector containing a halorhodopsin (AAV8-hSyn-JAWS-GFP) across all layers of macaque somatosensory cortex. The measurement bar in the lower left corner is 0.5 mm long. The stain corresponds to green-fluorescent protein (GFP) expression (and thus to opsin expression) and the red stain corresponds to neuronal expression (NeuN). The virus was injected at two sites (in this case, 1 μL per site) around which there is the most concentrated expression.

FIG. 1 shows that viral vectors can be used to express an opsin over large volumes of macaque cortex. Further, tracer studies have shown that intrinsic neuronal connections span millimeters of macaque cortex while electrophysiological studies show that the point image spread is up to 10 mm in certain brain areas (such as 7a) and at least a few millimeters in other visual areas. An exemplary tissue illuminator can modulate the light-activated particles, such as the proteins expressed in a large fraction of neurons (e.g., more than 80%) across large volumes in the primate cortex as disclosed below.

Omnidirectional Tissue Illumination for Medical Applications

Exemplary tissue illuminators may also be configured for use in vascular surgery (e.g., angioplasty), stent repair, laser cautery, dermatological surgery (e.g., treatment of cystic acne, tattoo removal), photo ablation, light-based uncaging of drugs, treatment of local, superficial infections, dentistry, gynecological applications, urological applications, etc. For instance, an exemplary tissue illuminator may be used to melt the plaque along the tip of a catheter. Because the tissue illuminator has a pointed tip, it can be advanced into the atherosclerotic plaque, then used to melt the plaque along its entire length simultaneously from the inside out. And because the tissue illuminator melts the plaque from the inside out, it can melt the plaque more quickly and/or at a lower temperature than in conventional laser angioplasty. The melted plaque may flow out of the area as soon as it melts, rather than remaining trapped until the entire blockage is melted as in conventional laser angioplasty. A tissue illuminator can also be used to melt atherosclerotic plaques that have formed inside of stents from the inside (lumen) out, which provides an advantage over starting the melting process at one end and getting re-solidification at the other. In otolaryngology, for example, the heat generated from the fiber could be used to melt severe impacted cerumen (ear wax).

An exemplary tissue illuminator's flexibility also reduces the likelihood of damage during insertion and the laser angioplasty. For instance, the illuminator's tip can be sharp enough to penetrate a plaque, but too flexible (not stiff enough) to penetrate the wall of a blood vessel. (Conversely, the flat tip of a conventional fiber cannot penetrate plaque, but a conventional fiber with a sharpened tip can easily penetrate a blood vessel wall.) In addition, the tissue illuminator can be used without the feedback circuitry or cooling units used in conventional catheters, so it may have a smaller diameter than other catheters.

Further, the tissue illuminator's diameter can be less than 100 microns at its tip, allowing the tissue illuminator to be used to melt plaque in microvasculature. A tissue illuminator can also be used to stop microbleeding from the microvasculature and from vessels that may be too small to be cauterized using conventional techniques by applying a relatively low level of intensity to close off small vessels over a larger area. An exemplary tissue illuminator can also be used to induce scarring over a small, localized area, at relatively low intensity.

A polymer fiber-based illuminator can also be used for dermatological applications. For instance, an illuminator could be fed into a skin lesion and UV light could be used to kill bacteria within the lesion, without drugs or with photo-activated drugs (e.g., in drug refractory infections in the respiratory tract, in patients with a contra-indication or allergy to the antibiotic regiment, or in localized infections in patients with decreased circulation, such as diabetic patients with infected foot ulcers). This fiber could be applied in other cosmetic applications (e.g., acne treatment, tattoo removal, cosmetic surgery, etc.), in any optomological applications where lasers are currently used, in urological and gynecologic treatments (e.g., of nodules or uterine fibroids), and in treatments for aliments along the gastrointestinal tract (e.g., hemorrhoids, polyps). Light from the tissue illuminator may also be used to heat tissue via absorption by resonant nanoparticles and/or cure adhesives used in dentistry, surgery, and other applications.

These illuminators may allow non-invasive light-based medical techniques to be employed invasively (e.g., in photodynamic therapy, light-based therapy for the treatment of internal scars, the treatment of warts with light, etc.) with minimal damage or risk to the patient. Further, an illuminator could be used wherever internal delivery of either heat or light is beneficial (e.g., photoactivation of light-caged drugs, photo-cauterization to stop bleeding in internal structures, illumination of light- or heat-sensitive structures to induce sensation via a prosthetic device, such as a cochlear implant, etc.). Outside of medicine, an exemplary illuminator could be of use anytime a narrow, tapered light delivery source is needed. For instance, it can be used to illuminate narrow spaces or lumens, such as tubing, for sensing, curing light-sensitive adhesives, or killing bacteria with ultraviolet light.

### Omnidirectional Tissue Illumination Systems

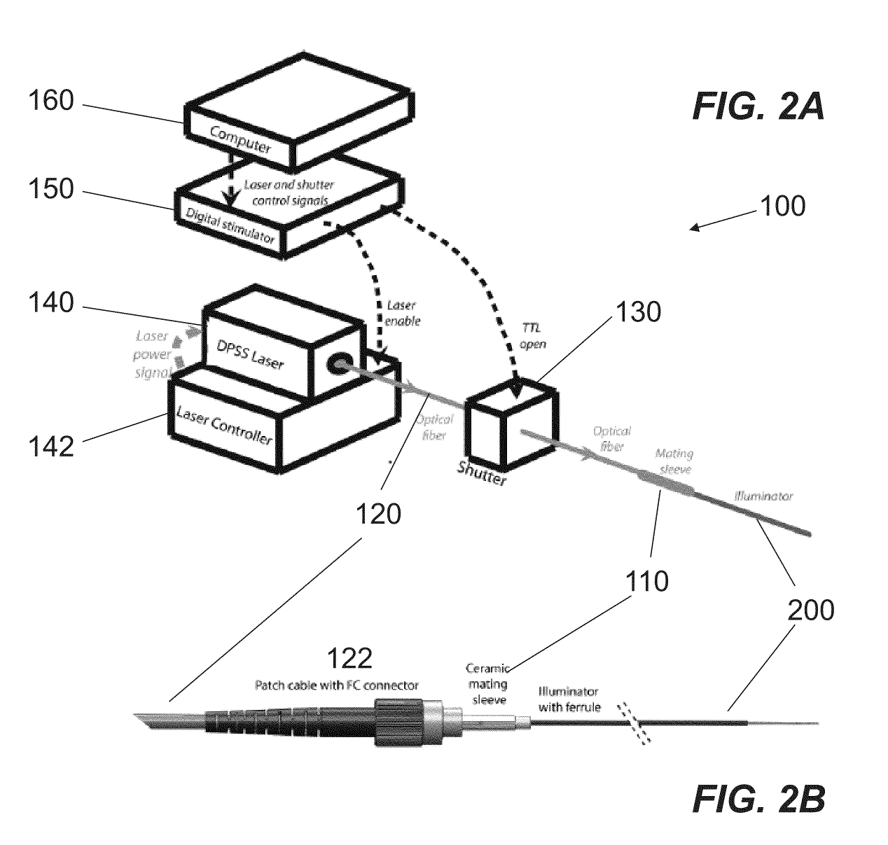

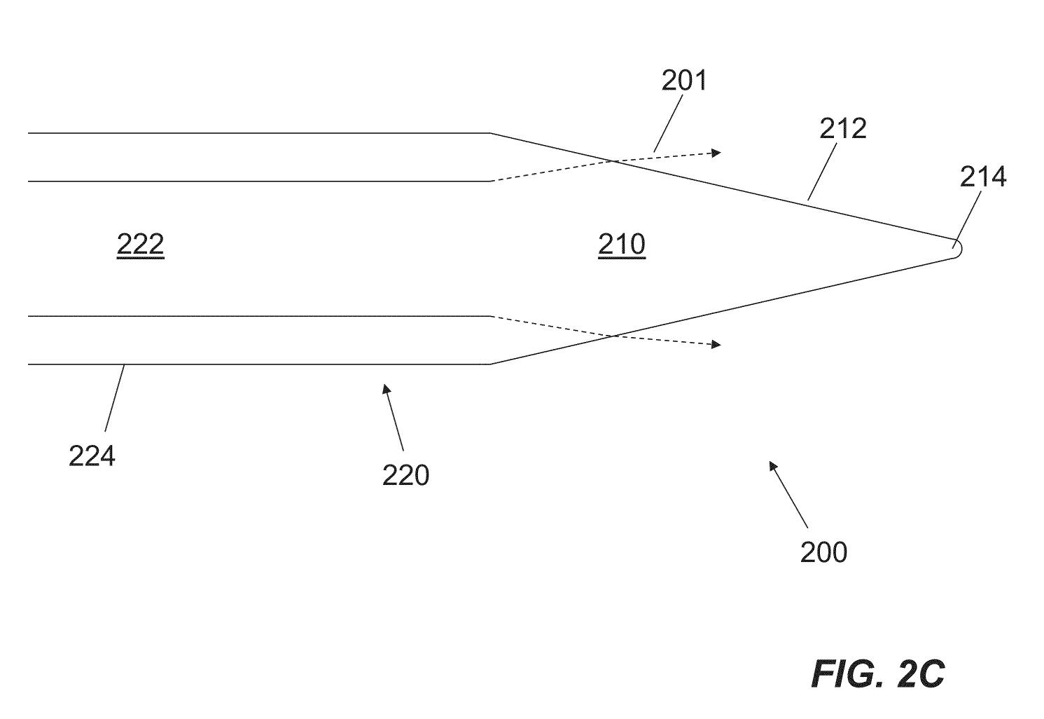

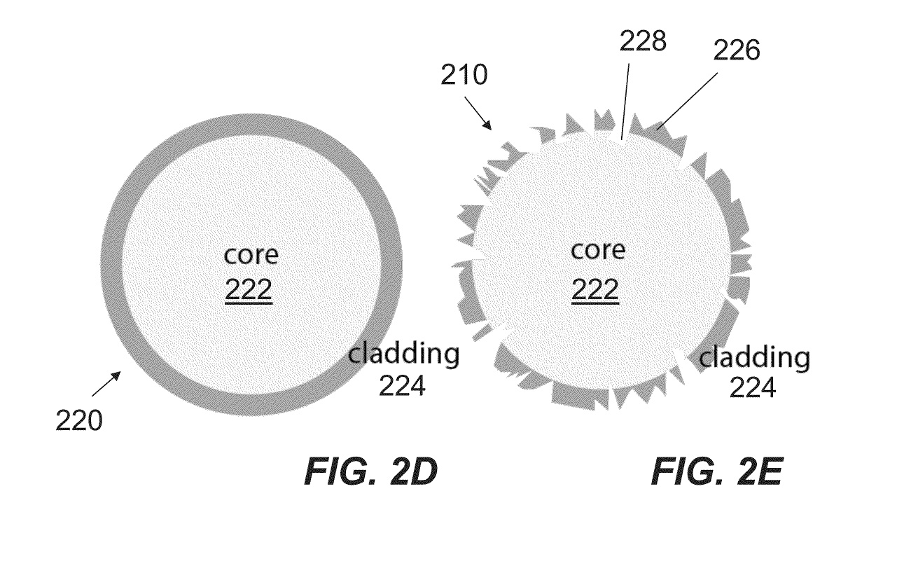

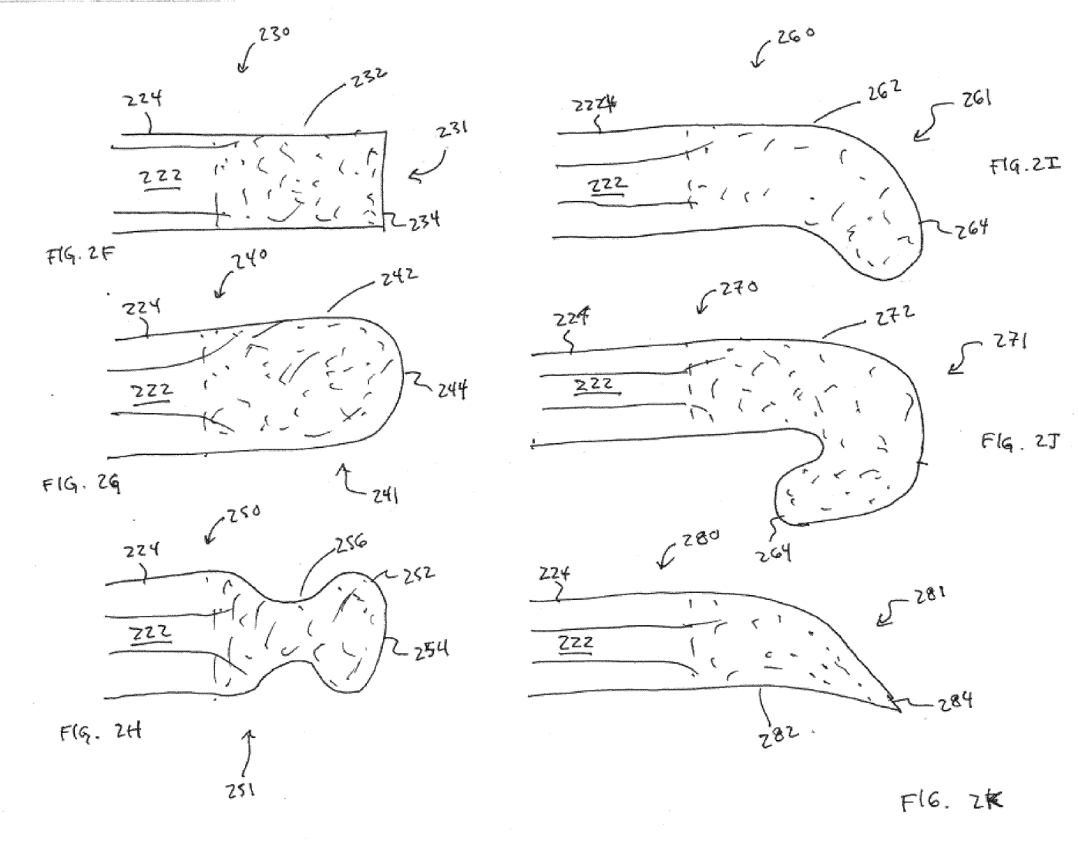

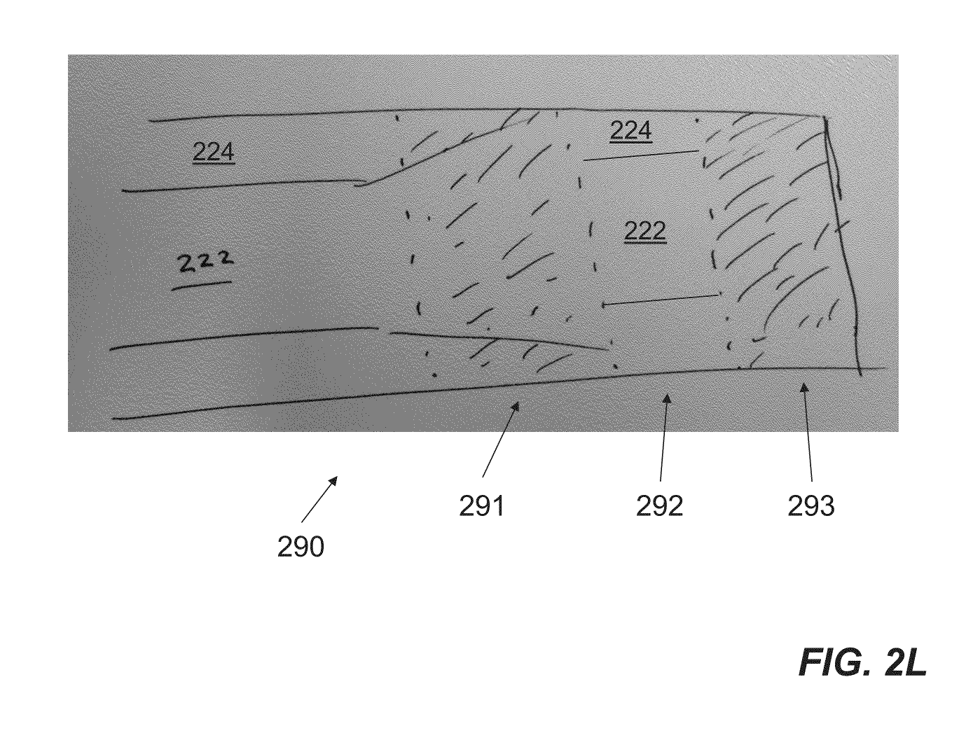

FIGS. 2A-2E illustrate an in vivo tissue illumination system 100 that includes a tissue illuminator 200 made of a flexible, biocompatible polymer or other suitable material. The system 100 includes a laser 140 (e.g., a diode-pumped solid state (DPSS) laser) or other suitable light source that is coupled to an optional fiber-coupled mechanical shutter 130 via a 200 tm diameter optical fiber 120. A computer 160 and a digital stimulator 150 control the shutter 130 and the laser 140. For instance, the shutter 130 may be opened or closed to transmit or block the laser beam in response to commands from the computer 160 and the digital stimulator 150. Another length of 200 tm diameter optical fiber 120 couples the shutter's output to the illuminator 200 through a ceramic mating sleeve 110.

Those of ordinary skill in the art will readily appreciate that other techniques and equipment may be used to modulate the beam emitted by the laser 140. For instance, the laser 140 may be modulated directly, e.g., by changing the drive current, to change to beam's intensity. Similarly, if the laser 140 is tunable, its output wavelength may be changed, e.g., by applying a voltage to a piezo-mounted grating or mirror that defines the laser cavity or by heating or cooling the laser cavity as well understood in the art.

The tissue illuminator 200 enables an increased illumination volume by emitting light over a larger surface area than conventional optical fiber. This decreases the irradiance at any given location for a given optical power level compared to emission through via a conventional optical fiber, which in turn may decrease tissue heating when used for in vivo applications. The tissue illuminator's tapered shape and thin tip 210 diameter reduce the risk of damaging tissue (compared to blunt fibers and other instruments) or causing other penetration damage, which is particularly beneficial for biological and medical applications. The large light-emitting surface 212 allows for more light to be delivered to a given volume of tissue than with conventional fibers, partially because less light is lost to absorption by propagation through intervening tissue. This reduced absorption may also lead to less tissue heating.

FIGS. 2C-2E show the tissue illuminator 200 in greater detail. The illuminator 200 includes an optical fiber 220, such as a plastic or polymer optical fiber, with a core 222 and a cladding 224 that guide light as well understood in the art. The optical fiber 220 may have a Young's modulus between about 1 GPa and 100 GPa (e.g., 1-5 GPa, 1.5-2.5 GPa, etc.), meaning that the fiber 220 is relatively flexible and thus less likely to damage tissue during insertion/penetration.

The optical fiber's distal end terminates in a tip 210 whose diameter narrows smoothly (e.g., linearly, such that the tip 210 is conical) to an apex 214. In some cases, the cone angle of the tip 210 may be about 1 degree to about 30 degrees (e.g., about 5 degrees, 10 degrees, 15 degrees, 20 degrees, or 25 degrees) to enhance penetration without unduly injuring tissue. The apex 214 may be pointed or slightly rounded—for instance, the apex 214 may have a radius of curvature from about 5 μm to about 100 μm. In addition, the apex's diameter may be much smaller than the illuminator's maximum diameter (e.g., an order of magnitude narrower or more at the tip)—for instance, a 250 micron diameter optical fiber with a 50 micron tip diameter—which leads to reduced penetration damage when the illuminator 200 is inserted into tissue (e.g., neural tissue).

FIGS. 2C and 2E also show that, in some embodiments, the tip 210 lacks a well-defined core 222 or cladding 224 like the optical fiber 220. Instead, the manufacturing process (described below) may cause the core to melt or diffuse into the cladding within the tip region, which causes the tip 210 to have a radial refractive index profile that may be smooth or arbitrarily varied rather than uniformly stepped like the optical fiber 220. Alternatively, or in addition, the cladding 224 may be abraded, etched, scored, or otherwise roughed to form one or more irregular (e.g., jagged) sections 226 that may be distributed irregularly, periodically, or aperiodically around the core's circumference. In some case, abrasions or etched regions 228 may extend into the core 222 itself. (By comparison, the cladding 224 is distributed uniformly about the core 222 of the optical fiber 220 as shown in FIG. 2D.) The variation in cladding distribution and/or radial refractive index profile cause light guided by the core 222 to diverge upon entering the tip 210, and then to exit the tip 210 as indicated by dashed line 201. The angular divergence may be equal to the fiber's numerical aperture and is determined by the core and cladding indices.

The diverging beam exits the tip 210 via the tip's outer surface 212, which may be etched or roughened to provide more diffuse illumination. For instance, when illuminated, the tip 210 may appear to be a Lambertian emitter, which has a radiant intensity that varies in proportion to the cosine of the angle between the surface normal and the viewer's line of sight. (In other cases, the light may be emitted perpendicular to the surface of the fiber, in which case the tip's emission may appear less than Lambertian.) In some embodiments, the emitted diffuse illumination may subtend a solid angle of about π/8 steradians to about π/2 steradians or more (e.g., up to π steradians).

To provide this diffuse illumination, the outer surface 212 is roughened or etched over a length extending from the apex 214 possibly to the junction of the tip 210 and the optical fiber 220. The outer surface 212 can be roughened on all sides (e.g., over the 360 degrees of the fiber's circumference), in a pattern, or just over a particular area or subsection. (Other sections of the optical fiber 220 may be drawn and roughened as well to provide additional illumination.)

Tissue Illuminators with Shaped Tips

FIGS. 2F-2K show tissue illuminators that provide diffuse illumination with tips of different shapes that are formed in the distal ends of respective multimode fibers, each of which has a core 222 and a cladding 224 as discussed above. FIG. 2F shows a tissue illuminator 230 with a blunt tip 231 that has a roughened cylindrical surface 232 concentric with the multimode fiber's longitudinal axis and roughened face 234 that is normal to the multimode fiber's longitudinal axis. This tissue illuminator 230 may be formed by cleaving a multimode fiber, then roughening its distal end as described below.

FIG. 2G shows a tissue illuminator 240 with a rounded tip 241 that has a roughened outer surface 242 with a curved portion 244 (e.g., curved in the shape of a hemisphere or a paraboloid). In operation, light radiates from the curved portion 244 of the rounded tip 241. This tissue illuminator 240 may be formed by cleaving a multimode fiber, softening the cleaved end (e.g., using an open flame or a fusion splicer), allowing the softened end to harden, and then roughening the distal end as described below.

FIG. 2H shows a tissue illuminator 250 with a bulbous tip 251 that has a roughened outer surface 252 with a curved portion 254 (e.g., curved in the shape of a sphere) that extends from a narrower neck 256. In operation, light radiates from the curved portion 254 and the neck 256. This tissue illuminator 250 may be formed by cleaving a multimode fiber, softening and pulling the cleaved end (e.g., using an open flame or a fusion splicer), allowing the softened end to harden, and then roughening the distal end as described below.

FIGS. 2I-2K show tissue illuminators with tips that are bent with respect to the multimode fiber's longitudinal axis. These bent tips may be inserted around obstructions, delicate regions, or tougher regions in tissue. They can also be used to secure the illuminators within the tissue, e.g., by hooking themselves in place to prevent inadvertent withdrawal.

FIG. 2I shows a shows a tissue illuminator 260 with a bent tip 261 that has a roughened outer surface 262 with a curved portion 264 (e.g., curved in the shape of a hemisphere or a paraboloid). In operation, light radiates from the curved portion 264 of the bent tip 261. This tissue illuminator 260 may be formed by cleaving a multimode fiber, softening and bending the cleaved end (e.g., using an open flame or a fusion splicer), allowing the softened end to harden, and then roughening the distal end as described below.

FIG. 2J shows a shows a tissue illuminator 270 with a flexible hooked tip 271 that has a roughened outer surface 272 with a curved portion 274 (e.g., curved in the shape of a hemisphere or a paraboloid). As shown in FIG. 2J, the hooked tip 271 extends along an arc of more than 90 degrees (e.g., 135 degrees, 180 degrees, etc.) when relaxed. This hooked tip 271 can be straightened, then loaded into a cannula (e.g., a 26 gauge guide needle) for insertion into tissue. Once the cannula is positioned appropriately, the fiber can be pushed out, causing the hooked tip 271 to spring back into its relaxed (hooked) shape and catch within the tissue. The tissue illuminator 270 can be withdrawn by reinserting the cannula and/or gently pulling on the multimode optical fiber until the hooked tip 271 straightens out. This tissue illuminator 270 may be formed by cleaving a multimode fiber, softening and bending the cleaved end (e.g., by heating the cleaved end while bending or wrapping it around a mandrel), allowing the softened end to harden, and then roughening the distal end as described below.

FIG. 2K shows a shows a tissue illuminator 280 with a bent, pointed tip 281 that has a roughened outer surface 282 with a curved portion 286 that terminates in a pointed apex 284. In operation, light radiates from the curved portion 284 of the bent tip 281. This tissue illuminator 280 may be formed by cleaving a multimode fiber, heating and pulling the fiber to form a pointed end, the softening and bending the pointed end (e.g., by heating the cleaved end while bending or wrapping it around a mandrel), allowing the pointed end to harden, and then roughening the distal end as described below.

FIG. 2L shows a shows a tissue illuminator 290 with a bent, pointed tip 281 that has a roughened outer surface 282 with a curved portion 286 that terminates in a pointed apex 284. In operation, light radiates from the curved portion 284 of the bent tip 281. This tissue illuminator 280 may be formed by cleaving a multimode fiber, heating and pulling the fiber to form a pointed end, the softening and bending the pointed end (e.g., by heating the cleaved end while bending or wrapping it around a mandrel), allowing the pointed end to harden, and then roughening the distal end as described below.

Diffuse Illumination from a Tissue Illuminator

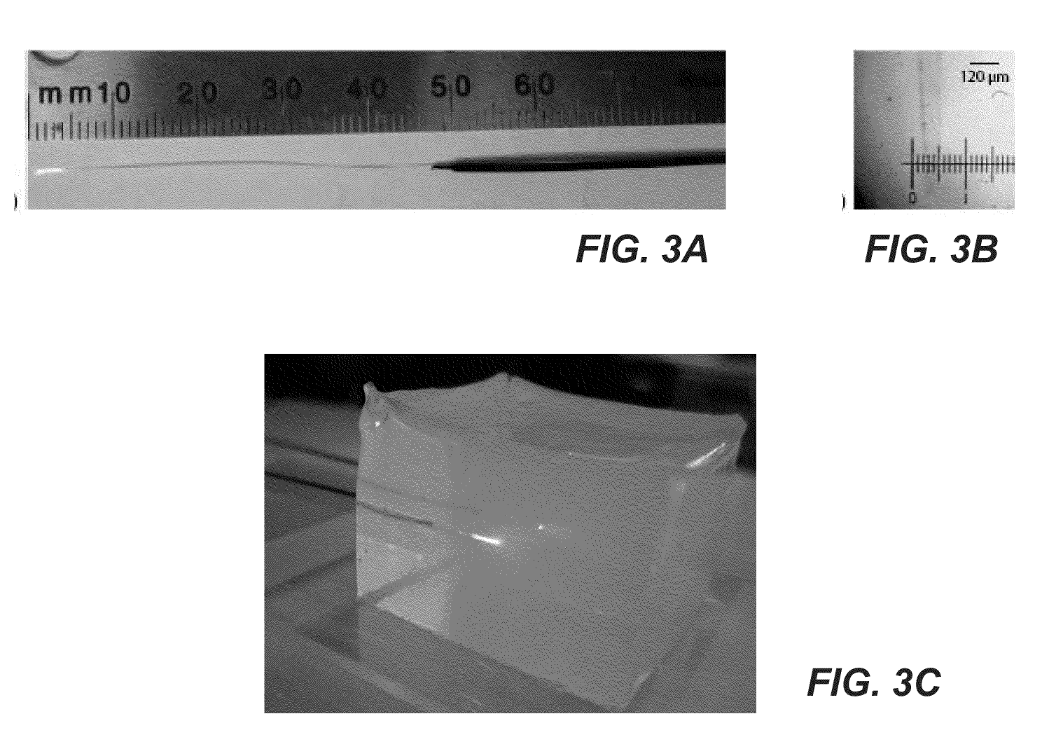

FIGS. 3A, 3B and 3C are photographs of an exemplary tissue illuminator on a table, in a close-up, and inserted into a block of agar, respectively. In FIG. 3C, the exemplary tissue illuminator appears in the foreground (closer to the observer) and a control device appears in the background. These devices both have the same total light power put into them, meaning that the exemplary tissue illuminator has a light power density at its surface which is one hundred times smaller than the light power density at the surface of the control illuminator, yet the exemplary tissue illuminator still spreads light over a larger area/volume. The exemplary illuminator has the same diameter as a standard optical fiber (a few hundred microns), but its light emitting surface area is on the order of square millimeters, not tens of square microns. With its larger light-emitting surface area, this illuminator can deliver light over 360 degrees along a 3 mm length, e.g., for optogenetic modulation in a primate cortex. For instance, the illuminator may be used to silence neurons injected with a halorhodopsin-containing viral vector along an entire injection track.

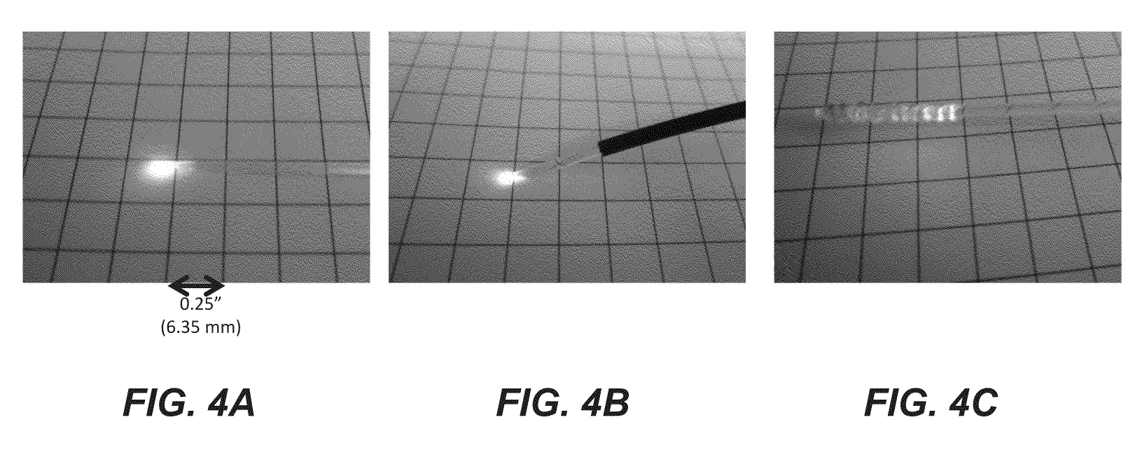

FIGS. 4A-4C are photographs of tissue illuminators with different tips emitting light at the same input wavelength and input power level. FIG. 4A shows a tissue illuminator with a conical tip, which emits more light than the tips shown in FIGS. 4B and 4C as measured with an integrating sphere as described below. FIG. 4B shows an illuminator with an angled tip that emits light from an angled facet and is relatively easy to fabricate. And FIG. 4C shows an illuminator with an etched tip that is easy to fabricate and emits diffuse illumination from all side over a relatively long distance (e.g., about 2 mm).

### Tissue Illuminator Construction

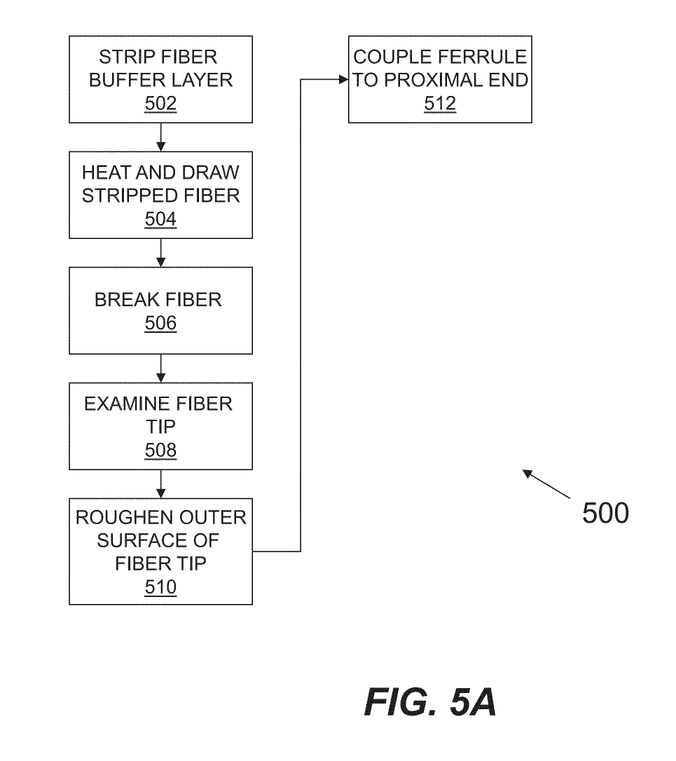

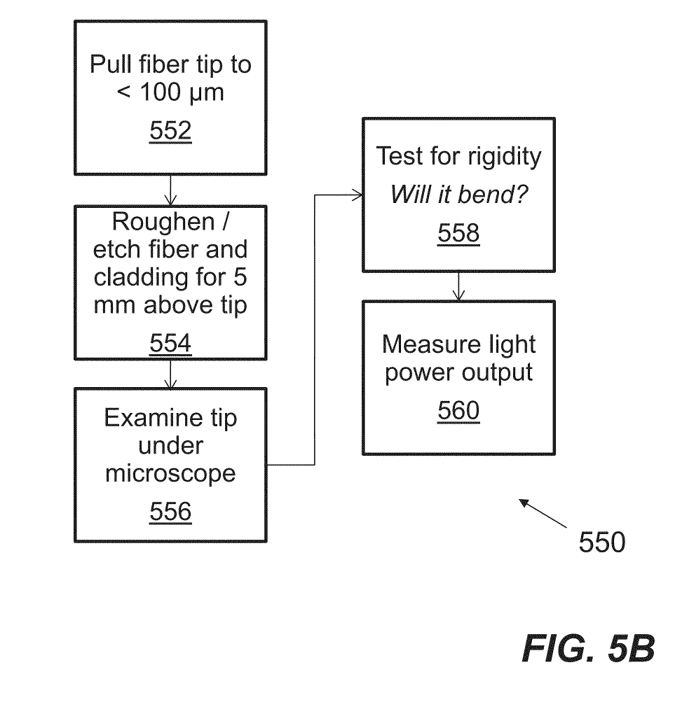

FIG. 5A illustrates a process 500 of making a tissue illuminator. Construction begins in step 502 with the removal of the buffer layer from one end of an optical fiber. For example, the buffer layer may be removed along about 15-20 cm from the end of a 250 μm plastic optical fiber with a 22-gauge wire stripper or other suitable device. In step 504, the optical fiber is heated and drawn to a predetermined thickness using a draw tower or other suitable equipment. When drawn by hand, the stripped end of the optical fiber may be locked in a vise, then pulled taut while the stripped section of the fiber is heated, e.g., using a dual temperature heat gun on the lower setting. Heating and pulling the fiber causes the heated section of the fiber to thin to a thickness of about 60 μm to about 100 μm. Once the heated section of the fiber reaches the desired thickness and shaped, the fiber may be allowed to cool, then pulled apart in step 506 at its thinnest point, e.g., by gripping about an inch to either side of the thinned portion. In step 508, the tip of the fiber is examined under a microscope or with another suitable device to verify that its shape is suitable for the desired application. For example, fibers with forked or curled tips may be discarded because they may be more likely to damage tissue than straight tips.

If the fiber has a suitably shaped tip (e.g., a conical or paraboloidal tip), it is etched in step 510. Etching may be done using an automatic process or by hand using any suitable combination of solvents, mechanical abrasion, photolithography, laser ablation (etching), thermal etching, and any other suitable roughening or etching technique. The desired portion of the fiber (e.g., the tip) may masked using tape or resist. For instance, tape may be applied to the fiber at a distance from the tip equal to the desired length of light emission. The exposed portion of the fiber is sanded first with a 5 μm grit sandpaper (e.g., a 5 μm Silicone Carbide Lapping Sheet from ThorLabs) and then with 3 μm grit sandpaper (e.g., a 3 μm Aluminum Oxide Lapping Sheet from ThorLabs) to create a uniform roughness on all sides of the fiber tip.

In step 512, the proximal end of the fiber is coupled to a ferrule. The outer buffer layer on the distal end of the fiber is exposed, e.g., using a 22-gauge wire stripper, then cleaved or cut flat and polished. The cleaved end of the fiber is glued to a stainless steel ferrule (e.g., with an inner diameter of 260 μm) using plastic epoxy or any other suitable adhesive. The epoxy is cured, excess epoxy is removed, and any protruding fiber is sanded off with polishing paper until the fiber is flush with the ferrule end. A dust cap may be used to protect the fiber from any debris or damage.

FIG. 5B is a flow diagram that illustrates a process 550 of fabricating tissue illuminators suitable for illuminating primate neural tissue. In step 552, the tip of an optical fiber is pulled to thin the fiber to a diameter of less than about 100 μm. The stretched fiber tip is roughened and/or etched over a length of about 5 mm from the distal end of the tip in step 554. The tip is then examined under a microscope or other suitable imager in step 556. If the tip's outer surface is too rough or not rough enough, the tip may be discarded or roughened further. If the examination shows that the tip's outer surface meets the desired roughness criteria, it is tested for rigidity in step 558. If the tip is too rigid or not rigid enough, it may be discarded. (The desired rigidity of the fiber can depend on the tissue that is being penetrated and the specific application for which the fiber is being designed, as with the other physical parameters (e.g., length of tip, taper angle, etc.).) Otherwise, if it meets the desired rigidity criteria, the tip's light output is measured in step 550.

Those of skill in the art will readily appreciate that the shape of the fiber tip may be affected by the drawing stress and fiber temperature used in step 504 (FIG. 5A) and step 552 (FIG. 5B). For instance, applying too much tension to the fiber will cause the fiber to snap and break while applying too little allows the fiber to curl upon itself. Similarly, the fiber should be heated enough so that it stretches without curling or breaking

Those of skill in the art will also appreciate the benefits of maintaining the mechanical integrity of the illuminator after fabrication. While glass or silica optical fibers may break when they are mechanically stressed or bent too sharply, plastic optical fibers typically do not break; rather, they may kink and emit light out of the kinked region. Though it is much easier to remove a kinked plastic fiber that remains in one piece than to remove shards of broken glass from tissue, both plastic optical fiber illuminators (such as this one) and glass fibers should be tested regularly to ensure that they have not sustained mechanical damage.

Illuminator Calibration

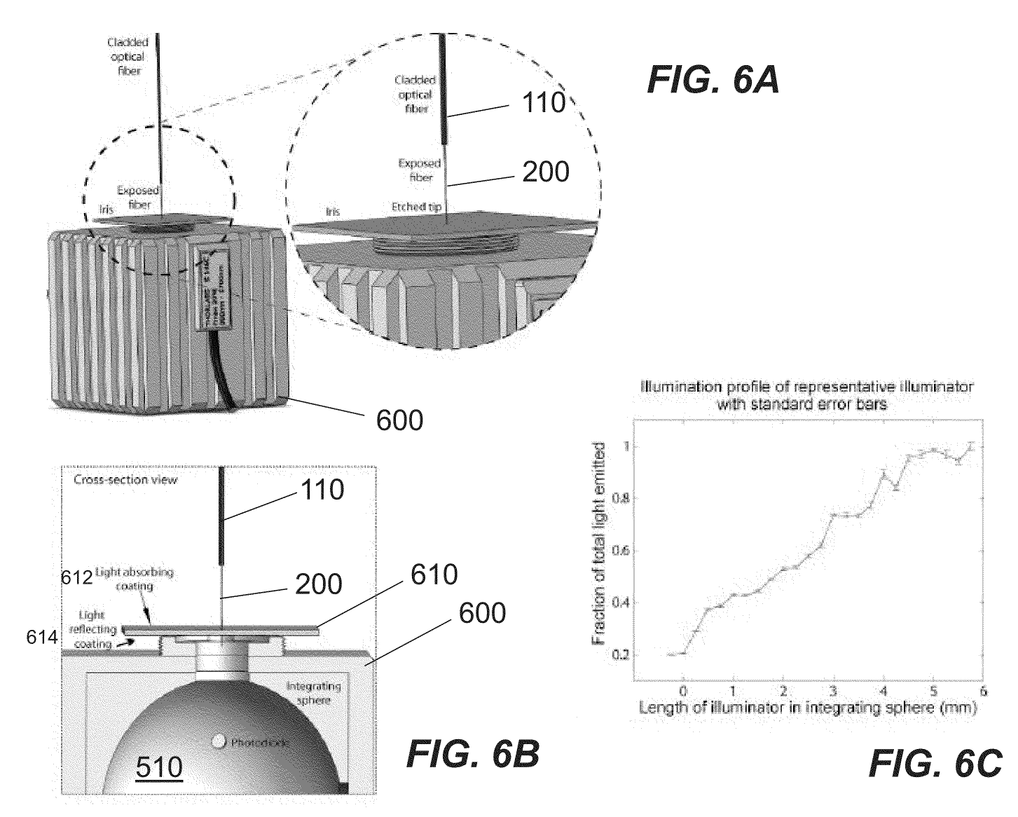

FIGS. 6A-6C illustrate calibration measurements of an exemplary tissue illuminator 200. The uniformity of light emission along the illuminator's etched fiber tip 220 (FIG. 2C) is measured via a lowering test, in which the illuminator 200 is lowered with a micromanipulator (not shown) in 500 tm increments into an integrating sphere 600 with custom shielding 610 at its opening. The custom shielding 610, with nonreflective material 612 on one side and reflective material 614 on the other, has a 26-gauge hole through with the penetrating illuminator 200 passes. Matching the diameter of the hole in the shielding to the diameter of the penetrating illuminator 220 reduces the amount of ambient light entering the integrating sphere 600.

FIG. 6C shows the total power output measured along the length of the illuminator 200 plotted as a function of the illuminator's depth in the integrating sphere 600. The plot shows that power varies linearly as function of insertion depth, which indicates that the illuminator emits light uniformly along it length.

### Exemplification

An exemplary tissue illuminator was used for optogenetic modulation of a portion of a male rhesus macaque's cortex. Results show that the illuminator illuminated a larger volume of the cortex with less penetration damage and thermal damage than might be expected with a conventional illuminator of similar size. Further, these results show that examples of the inventive illuminator can be used in the primate cortex to illuminate tissue along a length of at least 3 mm. The results also show that an exemplary illuminator can silence neurons expressing a halorhodopsin over this same 3 mm span, thus, facilitating optogenetic silencing of tissue volumes relevant in primate neuroscience.

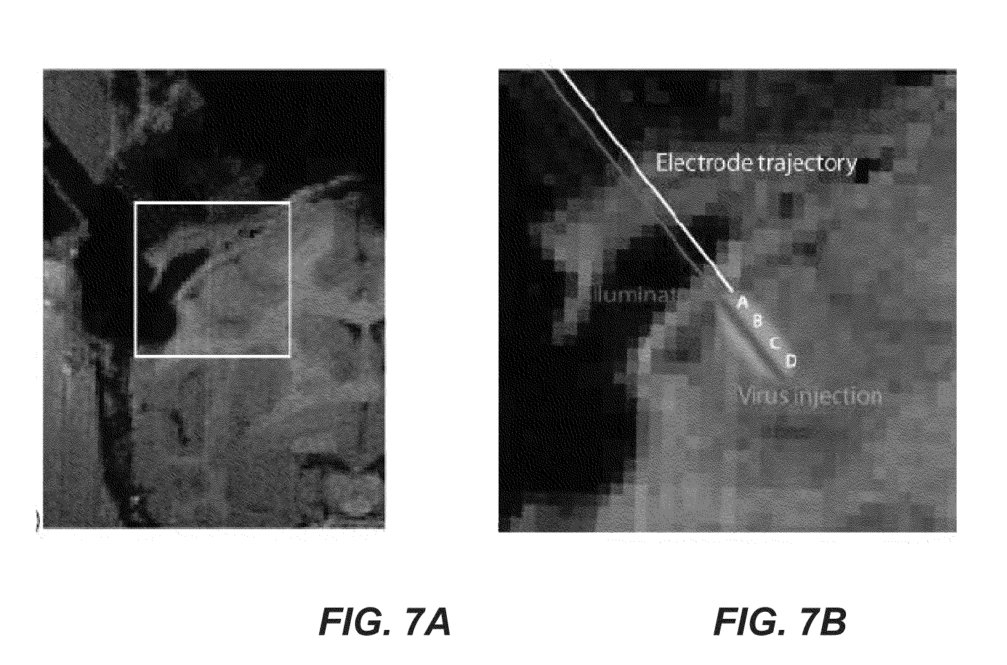

One male rhesus macaque weighing 13 kg was tested. The animal was cared for in accordance with the National Institutes of Health Guide for the Care and Use of Laboratory Animals and the guidelines of the MIT Animal Care and Use Committee. All surgical procedures were carried out under anesthesia. Antibiotics and analgesics were administered post-operatively, as needed. A recording well (19 mm inner diameter, Crist Instruments) over the frontal eye field and a titanium headpost were implanted surgically under aseptic conditions. Later an opsin-containing viral vector was stereotactically injected into cortex. The illuminator was not acutely placed in the cortex until six weeks post-injection. FIGS. 7A and 7B show the recording, illumination, and virus injection locations on an MRI image.

During all testing, the primate was head-fixed and seated in an ergonomically designed chair facing a computer screen. Two micromanipulators (NAN Instruments, Inc.) were mounted on the recording well. The fiber was lowered to the desired depth through a 25-gauge stainless steel guide tube held by one of the drives while a single-contact, parylene-coated tungsten microelectrode with approximately 1 MΩ resistance (WeSense) was lowered through another guide tube using the second drive. The illuminator remained in the same location throughout a recording session, but the electrode moved along its insertion trajectory. This enabled recording at different locations along the length of illumination. A custom grid was used to ensure that the fiber and electrode were lowered in parallel one millimeter apart from one another. The grid locked into place relative to the implanted recording well such that the virus injected sites could be accurately targeted months after injection. Prior to testing, it was visually confirmed that the primate was not exposed to any stray laser light. The entire implant and optics setup were shielded with light absorbing foil (ThorLabs) throughout testing.

The electrode was coupled to a single input channel on the pre-amplifier (Plexon) via a headstage (Plexon) and electrical connector (Omnetics). The pre-amplifier split the data into a spike channel (0.25-8 kHz) sampled at 40 kHz and a local field potential channel (0.7-170 Hz) sampled at 1 kHz. Pre-amplifier output went into a multi-channel acquisition processor, or MAP, box (Plexon) where it was filtered and acquired using the Rasputin (Plexon) software. Spikes were sorted offline using principle component analysis and manual waveform shape analysis (Offline Sorter, Plexon). MATLAB (Mathworks) was used for electrophysiological data analysis and plotting.

An optical artifact results when metal electrodes are illuminated with light, so the local field potential (LFP) signal was used to determine whether light was reaching a particular cortical location. Because this artifact is observed even in saline, it provides an illumination measure independent of opsin expression.

In this case, an optical artifact appeared in locations spanning a 3 mm length of cortex. The artifact is not uniform across either locations or recording sessions. Thus, using the optical artifact as a surrogate for measuring light power at a particular point in the tissue makes it possible to look for a change in the local field potential (LFP) that coincides with illumination, not for a particular shape or magnitude of deflection.

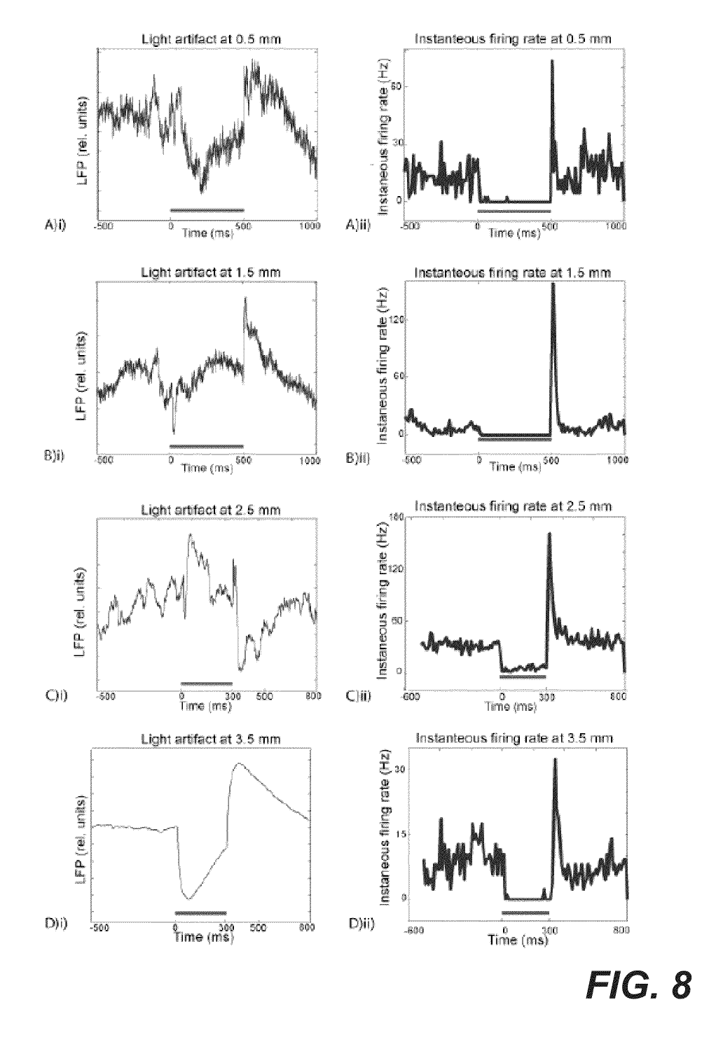

FIG. 8 shows the actual LFP deflection that occurred during illumination. The left column includes plots of the LFP recordings of representative neurons (left column) at the locations shown in FIGS. 7A and 7B and the right column includes instantaneous spike time histograms. The bar denotes when the laser is on. Note that the virus injections were performed at depths of about 1.0 mm relative to the surface of cortex to 3.5 mm relative to the surface of cortex in 0.5 mm intervals as shown in FIGS. 7A and 7B.

Illuminating neurons expressing a halorhodopsin with light of the appropriate wavelength suppresses neuronal firing and often leads to rebound excitation after illumination ceases. Illuminating the area that was previously injected with a viral vector containing a halorhodopsin yields both the expected neuronal suppression during the light pulse and rebound excitation. This is consistent with light reaching these neurons and corroborates the LFP findings for the same locations. FIG. 8 shows the instantaneous firing rate for four example neurons, spaced a millimeter apart from one another along the recording trajectory.

While this exemplification was performed with an illuminator having an etched, light-emitting surface about 3 mm to 5 mm in length, longer etched tips are possible for studies requiring illumination over longer trajectories and shorter tips could be manufactured for more focal illumination. Though this study uses 250 μm diameter fibers, thicker or thinner fibers could be used depending on the desired application. Further, one could potentially use non-uniform etching to create “dead spots” where no light is emitted. This would allow for light to be delivered, selectively to one layer of cortex or to one brain region, but not to another.

### Conclusion

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments can be implemented in any of numerous ways. For example, embodiments of designing and making the coupling structures and diffractive optical elements disclosed herein may be implemented using hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

The various methods or processes (e.g., of designing and making the coupling structures and diffractive optical elements disclosed above) outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An apparatus for providing diffuse illumination, the apparatus comprising:
a multimode optical fiber having:
a proximal end to couple light into the multimode optical fiber; and
a distal end terminating in a tapered tip that defines a roughened outer surface to emit at least a portion of the light coupled into the multimode optical fiber as diffuse illumination.

2. The apparatus of claim 1, wherein the multimode optical fiber comprises a polymer cladding disposed about a polymer core to guide the light from the proximal end to the distal end.

3. The apparatus of claim 1, wherein the multimode optical fiber has a stiffness of about 1 GPa to about 100 GPa.

4. The apparatus of claim 1, wherein the tapered tip has a maximum outer diameter of about 40 microns to about 5 millimeters and a length of about 1 millimeter to about 10 millimeters.

5. The apparatus of claim 1, wherein the tapered tip defines an apex forming an angle of about 1 degree to about 30 degrees.

6. The apparatus of claim 1, wherein the tapered tip is configured to be inserted into tissue at a reduced risk of damaging the tissue.

7. The apparatus of claim 1, wherein the tapered tip is configured to emit the diffuse illumination over a solid angle of about π/8 steradians to about π/2 steradians.

8. The apparatus of claim 1, wherein the roughened outer surface is formed by sanding at least a portion of the tapered tip with sandpaper having a grit of about 1 micron to about 5 microns.

9. The apparatus of claim 1, wherein the roughened outer surface has an area of about 0.1 mm² to about 100 mm².

10. The apparatus of claim 1, further comprising:
a light source, optically coupled to the proximal end, to emit the light into the multimode optical fiber.

11. The apparatus of claim 10, wherein the light source is configured to emit the light at a wavelength selected to modulate at least one light-activated particle disposed within an animal body.

12. The apparatus of claim 10, wherein the light source is configured to emit the light at a wavelength absorbed by tissue in an animal body.

13. A method of using an optical fiber to emit diffuse illumination within an animal body, the optical fiber having a proximal end and a distal end, the distal end terminating in a tapered tip having a roughened outer surface, the method comprising:
(A) inserting the distal end into the animal body;
(B) coupling light into the proximal end of the optical fiber;
(C) guiding the light from the proximal end to the distal end via the optical fiber; and
(D) emitting at least a portion of the light, via the roughened surface of the tapered tip, within the animal body as diffuse illumination.

14. The method of claim 13, wherein (A) comprises inserting the distal end into tissue of the animal body without substantially damaging vasculature in and/or surrounding the tissue.

15. The method of claim 14, wherein (A) further comprises guiding the tapered tip to a desired location within the tissue.

16. The method of claim 13, wherein (D) comprises emitting light at a wavelength selected to cure an adhesive disposed on and/or within the animal body.

17. The method of claim 13, further comprising:
(E) modulating at least one of a wavelength and an intensity of the light coupled into the proximal end of the optical fiber so as to modulate at least one light-activated particle illuminated by the diffuse illumination.

18. The method of claim 17, further comprising:
(F) recording a change in expression of at least one cell in response to modulation of the at least one light-activated protein.

19. The method of claim 13, wherein:
(A) comprises inserting the distal end into a lumen in the animal body; and
(D) comprises illuminating at least a portion of an interior surface of the lumen.

20. The method of claim 19, wherein (B) comprises generating the light at a wavelength absorbed by at least one of tissue of the lumen, plaque deposited on an interior surface of the lumen, a thrombus, a foreign object disposed within the animal body, a liquid injected into the animal body implanted material, a weakened region of tissue, scar tissue, a region of tissue deformation, and a cavity within the animal body.

21. The method of claim 19, further comprising:
(E) acquiring at least one image of the at least a portion of the interior surface of the lumen illuminated by the diffuse illumination.

22. The method of claim 13, wherein (D) comprises emitting the diffuse illumination over a solid angle of about π/8 steradians to about π/2 steradians.

23. The method of claim 13, wherein (D) comprises emitting the diffuse illumination through an area of the roughened surface of about 0.1 mm² to about 100 mm².

24. The method of claim 13, wherein (D) comprises illuminating an area within the animal body having a length of about 1 millimeter to about 10 millimeters.

25. A method of making a fiber-optic illuminator from an optical fiber, the method comprising:
(A) forming a distal end of the optical fiber into a tapered tip; and
(B) roughening at least a portion of the tapered tip so as to form a roughened outer surface that emits diffuse illumination when light is coupled into a proximal end of the optical fiber.

26. The method of claim 25, wherein (A) comprises forming the tapered tip to have a maximum outer diameter of about 40 microns to about 5 millimeters and a length of about 1 millimeter to about 10 millimeters.

27. The method of claim 25, wherein (A) comprises:
(A1) softening the distal end of the optical fiber; and
(A2) drawing the distal end of the optical fiber so as to form the tapered tip.

28. The method of claim 25, wherein (B) comprises at least one of:
(B1) mechanically abrading the at least a portion of the tapered tip;
(B2) chemically etching the at least a portion of the tapered tip;
(B3) laser etching the at least a portion of the tapered tip; and
(B4) thermally etching at least a portion of the tapered tip.

29. The method of claim 28, wherein (B1) comprises sanding the at least a portion of the tapered tip with sandpaper having a grit of about 1 micron to about 5 microns.

30. An apparatus for providing diffuse illumination, the apparatus comprising:
a multimode optical fiber having:
a proximal end to couple light into the multimode optical fiber; and
a distal end terminating in a tip having a curved, roughened outer surface to emit at least a portion of the light coupled into the multimode optical fiber as diffuse illumination.

31. The apparatus of claim 30, wherein at least a portion of the tip is bent with respect to a longitudinal axis of the multimode optical fiber.

32. The apparatus of claim 31, wherein the at least a portion of the tip is bent by at least about 90 degrees with respect to the longitudinal axis of the multimode optical fiber.
